(19) United States
(12) Patent Application Publication
(10) Pub. No.: US 2010/0167261 A1
(43) Pub. Date: Jul. 1, 2010

(54) PLATELET STABILIZATION

(75) Inventors: Josep Maria Jou, Barcelona (ES); Maribel Diaz-Ricart, Barcelona (ES); Gines Escolar, Barcelona (ES)

Correspondence Address: SIEMENS CORPORATION;INTELLECTUAL PROPERTY DEPARTMENT, 170 WOOD AVENUE SOUTH, ISELIN, NJ, 08830, US

(73) Assignee: SIEMENS HEALTHCARE DIAGNOSTICS INC., Tarrytown, NY (US)

(21) Appl. No.: 12/280,027

(22) PCT Filed: Feb. 20, 2007
(86) PCT No.: PCT/US2007/062408

§ 371 Date: Aug. 20, 2008

Related U.S. Application Data
(60) Provisional application No. 60/775,161, filed on Feb. 21, 2006.

(51) Int. Cl. A01N 1/02; C12M 1/00

(52) U.S. Cl. 435/2; 435/307.1

(57) ABSTRACT

Provided are methods for stabilizing mean platelet component and/or platelets in a sample comprising combining said sample with EDTA, a second anticoagulant, and one or more kinase inhibitors. Also provided are compositions for stabilizing a sample comprising platelets, and kits for storing a platelet sample.

### CROSS-REFERENCE TO RELATED APPLICATIONS

This claims benefit of U.S. Provisional Application No. 60/775,161, filed Feb. 21, 2006, the disclosure of which is hereby incorporated by reference in its entirety.

### FIELD OF THE INVENTION

The invention relates to the field of blood and/or platelet stabilization.

### BACKGROUND OF THE INVENTION

The ADVIA® 120 Hematology System is an automated analyzer that, in addition to measuring the conventional hematologic indices, also provides some activation-related information about platelets. Macey M G et al., Cytometry 38:250-55 (1999). It measures the intensity of light scattered by platelets at two different cone angles (2-3° and 5-15° and from the paired values computes platelet volume (PV) and platelet component (PC) concentration on a cell-by-cell basis. These values are then averaged to provide the Mean Platelet Component (MPC).

The MPC parameter calculated by the new generation of blood cell analyzers (ADVIA® 120 and 2120) provides direct information on density and granularity of platelets and indirect information on their structure and function. Macey M G et al.; Brummit D R & Barker H F, Clin. Lab Haematol. 22:103-107 (2000); Giacomini A et al., Clin. Lab Haematol. 23:181-186 (2001). Recent studies suggest that this parameter may be a useful indicator of in vitro and in vivo activation of platelets. Ahnadi C E et al., Thromb. Haemost. 90:940-48 (2003); Bae S H et al., Korean J. Opththalmol. 17:140-144 (2003); Chapman E S et al., Thromb. Haemost. 89:1004-1015 (2003); Giacomini A et al., Lab Hematol. 9:132-137 (2003); Ahnadi C E et al., Thromb. Haemost. 92:1207-1213 (2004).

Ethylenediaminetetraacetic acid (EDTA) is an anticoagulant currently used for blood cell counts and white blood cell differential analysis. Under optimal conditions, blood characteristics are well maintained from about one hour to about four hours after phlebotomy. While EDTA is an optimal anticoagulant for cell counting and white blood cell differential analysis, several studies point out that that anticoagulant is far from optimal for the preservation of platelet ultrastructural and functional capabilities. See White J G, Platelets 11:49-55 (2000); White J G & Escolar G, Platelets 11:56-61 (2000); White J G et al., Platelets 10:327-337 (1999); White J G, Scand. J. Haematol. 5:241-254 (1968). For example, it is well established that exposure to EDTA for prolonged periods results in extreme distortion of platelet morphology, including dilation of the open canalicular system (OCS) and a progressive tendency to sphericity. White J G & Escolar G (cited supra); White J G et al. (cited supra); White J G, Scand. J. Haematol. 5:241-254 (cited supra); Zucker M B & Borrelli J, Blood 9:602-608 (1954); Gachet C et al., J. Cell. Biol. 120:1021-1030 (1993). Severe calcium deprivation caused by EDTA would be responsible for dissociation of GPIIb-IIIa complexes, but also for alterations in the platelet OCS that isolate more internal segments of the OCS from the external milieu. Gachet C et al.; Woods V L et al., J. Biol. Chem. 261:15242-15251 (1986).

In view of the deleterious effects of EDTA on platelets, it is not unexpected that the mean platelet component has been found to be greatly affected by time and storage conditions, and under certain conditions is a less reliable parameter after three hours following sample collection. Macey M G et al.; Macey M Clin. Chem. 48:891-899 (2002). Although clinical storage and blood transportation logistics require platelet stability for up to six hours, suitable replacements for the current EDTA blood collection regime have not yet been found.

### SUMMARY OF THE INVENTION

The present invention is directed to compositions that permit prolonged blood and platelet stabilization under standard clinical conditions, methods for stabilizing blood and platelet samples, and compositions and kits useful for the storage of blood and platelet samples.

Disclosed are methods comprising combining a sample comprising platelets with an anticoagulant comprising EDTA and one or more kinase inhibitors, thereby forming a combination, wherein the mean platelet component of the sample is stabilized. Also disclosed are methods comprising combining a sample comprising platelets with an anticoagulant comprising EDTA and one or more kinase inhibitors, thereby forming a combination, wherein the platelets in the sample are stabilized.

Also provided are compositions for stabilizing samples comprising platelets comprising an anticoagulant comprising EDTA and one or more kinase inhibitors.

The present invention is also directed to kits for storing a platelet sample comprising a vessel for said sample, and storage reagents including an anticoagulant comprising EDTA and one or more kinase inhibitors.

### BRIEF DESCRIPTION OF THE DRAWINGS

The summary, as well as the following detailed description, is further understood when read in conjunction with the appended figures. For the purpose of illustrating the invention, there are shown in the figures exemplary embodiments of the invention; however, the invention is not limited to the specific methods, compositions, and characteristics disclosed.

FIG. 1 provides graphical data for experiments evaluating the effects on the mean platelet component (MPC) of storing samples comprising platelets in EDTA alone versus storage of samples using embodiments of the inventive methods.

FIG. 2 provides graphical data for experiments evaluating the effects on the mean platelet value (MPV) of storing samples comprising platelets in EDTA alone versus storage of samples using embodiments of the inventive methods.

FIG. 3 provides graphical data for experiments evaluating the effects on P-selectin expression of storing samples comprising platelets in EDTA alone versus storage of samples using embodiments of the inventive methods.

FIG. 4 provides graphical results of a morphometric evaluation of the number of α-granules per platelet using electron microscopy cross sections of samples comprising platelets stored in EDTA alone as compared with platelets stored in accordance with an embodiment of the inventive methods.

FIG. 5 is an electron microscopy morphological analysis of platelets stored in EDTA alone as compared with platelets stored in accordance with an embodiment of the inventive methods.

FIG. 6 depicts high-magnification images of platelets stored in EDTA alone and stored in accordance with two embodiments of the inventive methods pursuant to an evaluation of α-granule characteristics.

FIG. 7 provides a plot of the mean number of dense bodies (also referred to as dense granules) per platelet measured in platelet samples stored in EDTA or STABILE-1 formulation for designated time periods.

FIG. 8 is box-and-whisker plot of the mean number of dense bodies per platelet measured in platelet samples stored in EDTA or STABILE-1 formulation for designated time periods.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “an anticoagulant” is a reference to one or more of such anticoagulants and equivalents thereof known to those skilled in the art, and so forth. When values are expressed as approximations, by use of the antecedent “about,” it will be understood that the particular value forms another embodiment. As used herein, “about X” (where X is a numerical value) refers to ±10% of the recited value, inclusive. For example, the phrase “about 8” refers to a value of 7.2 to 8.8, inclusive; as another example, the phrase “about 8%” refers to a value of 7.2% to 8.8%, inclusive. Where present, all ranges are inclusive and combinable.

Newly discovered formulations are capable of stabilizing platelets in biological samples for periods of time that exceed that which is provided by current blood storage reagents. Prior to the present invention, strategies based on the addition of low concentrations of fixatives (for example, paraformaldehyde or glutaraldehyde), while proving useful for flow cytometry, have not been shown to stabilize MPC. Likewise, storage in current anticoagulants based on EDTA result in progressive increase in MPV and P-selectin expression, a decrease in MPC, and a significant reduction in the number of platelet α-granules and dense bodies. In contrast, methods based on the present formulations provide reliable stability for the MPC parameter for periods of time exceeding six hours following blood collection. The inventive formulations can also stabilize other blood parameters, including mean platelet volume (MPV) and/or P-selectin expression, and can also or alternatively prevent the reduction of platelet α-granules and/or dense bodies, for an amount of time that is consistent with the requirements of typical clinical storage and blood transportation procedures.

As used herein, the term “anticoagulant” refers to a substances that delays, slows, retards, suppresses, minimizes, prevents, or inhibits the clotting or coagulation of blood or a component thereof.

Disclosed are methods comprising combining a sample comprising platelets with an anticoagulant comprising EDTA and one or more kinase inhibitors, thereby forming a combination, wherein the platelets in the sample are stabilized. In preferred embodiments, the combination further comprises a second anticoagulant.

Also provided are compositions for stabilizing a sample comprising platelets comprising an anticoagulant comprising EDTA and one or more kinase inhibitors.

As used herein, the term “anticoagulant” refers to one or more substances that delay, slow, retard, suppress, reduce, minimize, prevent, and/or inhibit the clotting or coagulation of blood or a component thereof.

Also as used herein, the term “combination” or “combining” or derivatives thereof refers to a collection of substances that have been combined via any physical and/or chemical process, or to the act of forming a collection of substances via any physical and/or chemical process.

“Stabilization” refers to the effect of completely or partially preventing or reducing deviation from a starting, baseline, or ideal value; for example, stabilization of the mean platelet component can refer to the effect of reducing deviation of the mean platelet component value at time t=6 hr from the baseline mean platelet component value measured at a starting time.

A “kinase inhibitor” is a substance that tends directly or indirectly to alter the activity of a protein kinase.

A “sample comprising platelets” is a material that includes platelets or components thereof. Nonlimiting examples of samples comprising platelets include human or animal blood.

Preferably, the combination of the sample comprising platelets, EDTA, the optional second anticoagulant, and one or more kinase inhibitors is effected by adding these substances to a vessel, such as a sample collection tube. The sample collection tube can be a blood collection tube, many types of which are commercially available. For example, the sample collection tube can be a Becton Dickinson Vacutainer® blood collection tube (Becton, Dickinson and Company, Franklin Lakes, N.J.). In exemplary embodiments, the sample comprising platelets is collected into a sample collection vessel, and the EDTA, the optional second anticoagulant, and one or more kinase inhibitors are subsequently added. In other instances, the EDTA, optional second anticoagulant, and one or more kinase inhibitors are added to a sample collection vessel, into which the sample comprising platelets is subsequently introduced. The sample collection vessel can be a commercially available EDTA tube that is pre-filled with EDTA, and in such instances, the other members of the combination are added to such pre-filled tube. The instant invention encompasses the combination of a sample comprising platelets, EDTA, an optional second anticoagulant, and one or more kinase inhibitors in any proportion, order, sequence, or progression.

The elapsed time between the collection of the sample comprising platelets and the combining of the EDTA, an optional second anticoagulant, and one or more kinase inhibitors therewith is ideally brief, and preferably occurs over a matter of minutes, and even more preferably over a matter of seconds. Thus, for example, a sample comprising platelets may comprise blood that is collected from a subject at a starting time, and the combining of the blood with the EDTA, an optional second anticoagulant, and one or more kinase inhibitors can occur within about five minutes following such starting time. Preferably, the combining of the sample comprising platelets with the other members of the combination occurs within about three minutes or less following the acquisition of the sample comprising platelets, although longer periods of time are also contemplated as being within the scope of the instant invention. Gentle mixing of the resulting combination, for example, by carefully inverting the sample collection tube one or more times (inverting the tube seven to eight times being a preferred embodiment), preferably follows the combining step. The combination may subsequently be subjected to analysis using the ADVIA® 120 or 2120 Hematology System (Bayer AG, Leverkusen, Germany). Processing of the combination through the ADVIA® system can take an additional period of time, preferably not more than about five minutes.

Following the combining step, the combination can be stored, for example, pending subsequent use. The inventive methods permit storage of samples comprising platelets for periods of time commensurate with the delay that is typically associated with, for example, transfer of samples from one medical facility to another, arrival of necessary medical personnel, or acquisition of equipment or materials needed for subsequent testing or processing of such samples. Long term storage of platelets using only EDTA or other storage methodologies known in the art is associated with progressive increase in MPV and P-selectin, a decrease in MPC, and a significant reduction in the number of platelet α-granules and dense bodies. Macey M et al., Clin. Chem. 48:891-899 (2002). In contrast, the present methods result in the stabilization of samples comprising platelets as characterized by stabilization of one or more of MPC, MPV, P-selectin, α-granules, and dense bodies for extended time periods.

Accordingly, the inventive methods can further comprise measuring mean platelet component of the stabilized sample comprising platelets. Alternatively or additionally, current methods can comprise measuring one or more of mean platelet volume (MPV), P-selectin, α-granules, and dense bodies of the stabilized sample comprising platelets. As described herein, the MPC, MPV, and P-selectin parameters can be measured using the ADVIA® 120 or 2120 Hematology System (Bayer AG, Leverkusen, Germany). Alpha (α)-granules and dense bodies can be counted using electron microscopy (EM) analysis. See, e.g., Joseph R et al., Stroke. 20(10:1316-9 (1989). As described above, baseline or initial values for one or more of MPC, MPV, P-selectin, α-granules, and dense bodies can be measured shortly after the combination of the sample comprising platelets, the EDTA, an optional second anticoagulant, and one or more kinase inhibitors, which is designated time t=0. In other embodiments, the baseline values for one or more of MPC, MPV, P-selectin, α-granules, and dense bodies are measured 30 min after the combination of the sample comprising platelets, the EDTA, an optional second anticoagulant, and one or more kinase inhibitors; this is designated time t=0+30. The sample comprising platelets can be stored for up to about one hour, up to three hours, up to about six hours, up to about eight hours, or up to about 24 hours following the combination of the sample comprising platelets with the EDTA, option second anticoagulant, and one or more kinase inhibitors. Preferably, the sample comprising platelets is stored at about 22° C. or at about 4° C. As provided previously, the elapsed time between the collection of the sample comprising platelets and the combining of the EDTA, an optional second anticoagulant, and one or more kinase inhibitors therewith is ideally brief, and preferably occurs over a matter of minutes.

When the sample comprising platelets is stored for up to about three hours at 22° C., and where the about three-hour storage period consists of the amount of time that elapses following the initial combination step, the use of the present invention affords a decrease in the mean platelet component by about 6% or less from the mean platelet component value at t=0. Also under these conditions, the use of the present invention affords an increase in the mean platelet volume (MPV) by about 8% or less from the MPV at t=0.

When the sample comprising platelets is stored for up to about six hours at 22° C., and where the about six-hour storage period consists of the amount of time that elapses following the initial combination step, the use of the present invention affords a decrease in the mean platelet component by about 10% or less from the mean platelet component value at t=0. Also under these conditions, the use of the present invention affords an increase in the mean platelet volume (MPV) by about 10% or less from the MPV at t=0.

In other embodiments, when the sample comprising platelets is stored for up to about six hours at 22° C., and where the about six hour storage period consists of the amount of time that elapses following the initial combination step, the use of the present invention affords a decrease in the mean platelet component by about 7% or less from the mean platelet component value at t=0+30 (i.e., from the mean platelet component value at 30 min following the initial combination step). Also under these conditions and using the aforementioned time points, the use of the present invention affords an increase in the mean platelet volume (MPV) by about 8% or less from the MPV at t=0+30, and also affords an increase in P-selectin by about 12% or less from the P-selectin value at t=0+30.

In other embodiments, when the sample comprising platelets is stored for up to about six hours at 4° C., and where the about six hour storage period consists of the amount of time that elapses following the initial combination step, the use of the present invention affords a decrease in the mean platelet component by about 7% or less from the mean platelet component value at t=0+30 (i.e., from the mean platelet component value at 30 min following the initial combination step). Also under these conditions and using the aforementioned time points, the use of the present invention affords an increase in the mean platelet volume (MPV) by about 8% or less from the MPV at t=0+30, and also affords an increase in P-selectin by about 12% or less from the P-selectin value at t=0+30.

In certain embodiments of the instant methods and compositions, the one or more kinase inhibitors is a phosphatidylinositol kinase inhibitor. Wortmanin, tyrphostin, or combinations thereof are preferred for use as the one or more kinase inhibitors. In highly preferred embodiments, the tyrphostin is tyrphostin 47. Wortmanin and tyrphostin (including tyrphostin 47) are each readily commercially available, e.g., from Sigma-Aldrich Corp. (St. Louis, Mo.).

When the one or more kinase inhibitors comprises wortmanin, the final concentration of wortmanin in the combination can be about 0.5 μM to about 2 μM; in other embodiments, the final concentration of wortmanin in the combination can be about 0.7 μM to about 1.5 μM; in preferred embodiments, the final concentration of wortmanin in the combination is about 1 μM.

When the one or more kinase inhibitors comprises tyrphostin, the final concentration of tyrphostin in the combination can be from about 25 μM to about 100 μM; in other embodiments, the final concentration of tyrphostin in the combination can be from about 35 μM to about 80 μM; in preferred embodiments, the final concentration of tyrphostin in the combination is about 50 μM.

When the one or more kinase inhibitors comprises both wortmanin and tyrphostin, which represents a highly preferred embodiment, the final concentration of wortmanin in the combination can be about 0.5 μM to about 2 μM and the final concentration of tyrphostin in said combination can be from about 25 μM to about 100 μM; in other embodiments, the final concentration of wortmanin in the combination can be about 0.7 μM to about 1.5 μM and the final concentration of tyrphostin in said combination can be from about 35 μM to about 80 μM; in preferred embodiments, the final concentration of wortmanin in the combination is about 1 μM and the final concentration of tyrphostin in said combination is about 50 μM.

Where the combination of a sample comprising platelets, EDTA, and one or more kinase inhibitors further comprises a second anticoagulant, or where the disclosed compositions further comprise a second anticoagulant, the second anticoagulant can comprise citrate. The citrate anticoagulant is preferably used in the form of a citrate-phosphate-dextrose composition. When a citrate, phosphate, and dextrose composition is used, the citrate can comprise trisodium citrate 2H₂O and citric acid H₂O, and the phosphate can comprise sodium hydrogen phosphate. The final concentration of the trisodium citrate 2H₂O in the combination can be from about 0.5 mM to about 2.5 mM, the final concentration of the citric acid H₂O in the combination can be about 0.1 mM to about 0.5 mM, and the final concentration of dextrose in the combination can be about 0.4 mM to about 1.6 mM. In preferred embodiments wherein the citrate anticoagulant is a composition of citrate, phosphate, and dextrose, the final concentrations in the combination are as follows: about 1.1 mM trisodium citrate 2H₂O; about 0.2 mM citric acid H₂O; about 0.2 mM sodium hydrogen phosphate; and, about 0.8 mM dextrose.

In the present methods and compositions, the anticoagulant comprising EDTA can comprise EDTA, dipotassium EDTA (K₂EDTA), or tripotassium EDTA (K₃EDTA). Anticoagulants comprising EDTA are readily available from commercial sources, for example, as bulk reagents or in aliquots distributed into individual sample-collection vessels. In preferred embodiments, the anticoagulant comprising EDTA is tripotassium EDTA. The final concentration of the tripotassium EDTA in the combination can be from about 2.0 mM to about 9.5 mM. K₃EDTA is available, for example, in individual aliquots from Becton, Dickinson and Company, Franklin Lakes, N.J. (Vacutainer® K3E 4.5 mL, Cat. No. 367654).

As used herein, the term “STABILE-1” refers to an embodiment of the instant invention in which a formulation comprising K₃EDTA, citrate-phosphate-dextrose, wortmanin, and tyrphostin is used. The STABILE-1 formulation includes the above-referenced components in the following concentrations when all components have been combined with the sample comprising platelets:

| | | |
|---|---|---|
| K₃EDTA | 4.64 | mM |
| wortmanin | 1.30 | μM |
| tyrphostin 47 | 50.45 | μM |
| trisodium citrate 2H₂O | 1.111 | mM |
| citric acid H₂O | 0.177 | mM |
| sodium hydrogen phosphate | 0.2 | mM |
| dextrose | 0.75 | mM |

The instant invention additionally comprises kits for storing a sample comprising platelets. The kits can comprise a vessel for the sample comprising platelets and storage reagents comprising an anticoagulant comprising EDTA, and one or more kinase inhibitors. The vessel for the sample comprising platelets ideally comprises a sample collection tube, although other types of vessels are also contemplated herein. The sample collection tube can be a blood collection tube, many types of which are commercially available. For example, the sample collection tube can be a Becton Dickinson Vacutainer® blood collection tube (Becton, Dickinson and Company, Franklin Lakes, N.J.). The instant kits can further comprise instructions for using the kit.

In certain embodiments of disclosed kits, the one or more kinase inhibitors is a phosphatidylinositol kinase inhibitor. Wortmanin, tyrphostin, or combinations thereof are preferred for use as the one or more kinase inhibitors. Tyrphostin 47 is a preferred form of tyrphostin.

The current kits can further comprise a second anticoagulant. Where the inventive kit further comprises a second anticoagulant, the second anticoagulant can comprise citrate. The citrate anticoagulant is preferably used in the form of a citrate-phosphate-dextrose composition. When a citrate, phosphate, and dextrose composition is used, the citrate can comprise trisodium citrate 2H₂O and citric acid H₂O, and the phosphate can comprise sodium hydrogen phosphate.

The anticoagulant comprising EDTA can comprise EDTA, dipotassium EDTA (K₂EDTA), or tripotassium EDTA (K₃EDTA). In preferred embodiments, the anticoagulant comprising EDTA is tripotassium EDTA.

The present invention is further defined in the Examples included herein. It should be understood that these examples, while indicating preferred embodiments of the invention, are given by way of illustration only, and should not be construed as limiting the appended claims From the present disclosure and these examples, one skilled in the art can ascertain the essential characteristics of this invention, and without departing from the spirit and scope thereof, can make various changes and modifications of the invention to adapt it to various usages and conditions.

### EXAMPLES

### Example 1

### Stabilization of MPC and MPV

Blood from healthy controls were carefully collected into standard sample collection tubes containing EDTA and stored at room temperature. The tubes were then gently agitated, and mean platelet component and mean platelet volume values were obtained from the combined blood/EDTA sample at the designated time points using the ADVIA® 2120 Hematology System (Bayer AG, Leverkusen, Germany) Blood from healthy controls were also collected into Vacutainer® K3E 4.5 mL K₃EDTA sample collection tubes (Cat. No. 367654, Becton, Dickinson and Company, Franklin Lakes, N.J.) and combined with the remaining STABILE-1 reagents (citrate-phosphate-dextrose, wortmanin, tyrphostin) to yield the prescribed concentrations. The samples were subjected to gently agitation, and were stored either at room temperature (22° C.) or at 4° C., and mean platelet component and mean platelet volume values were obtained at the designated time points using the ADVIA® 2120 system.

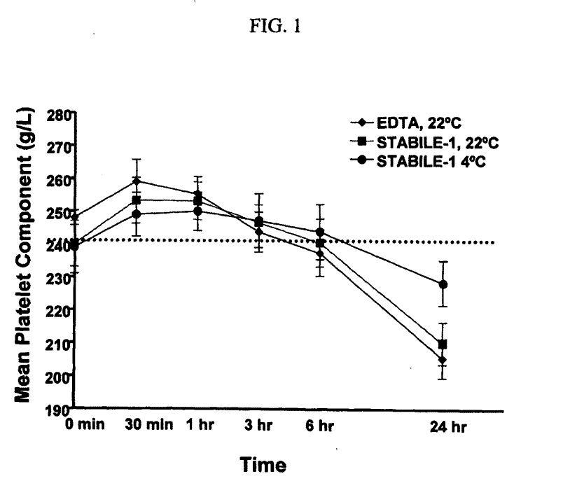

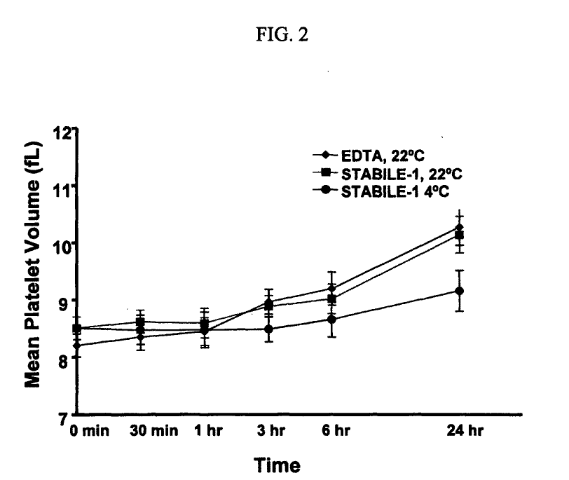

FIGS. 1 and 2 provide graphical data for experiments during which the mean platelet component and MPV, respectively, were measured during the storage of samples comprising platelets in 1) EDTA at 22° C.; 2) STABILE-1 formulation, stored at 22° C.; and, 3) STABILE-1 formulation, stored at 4° C. Data points are provided at 30 min, 1 hr, 3 hr, 6 hr, and 24 hr following the initial combination of the storage medium and the samples comprising platelets.

FIGS. 1 and 2 demonstrate that the STABILE-1 formulation, both when used under room temperature (22° C.) storage conditions and when used at 4° C., provides enhanced stabilization of the mean platelet component (MPC) and mean platelet volume (MPV), respectively, in samples comprising platelets as compared with the prior art EDTA storage medium. In particular, the decrease of MPC and increase of MPV is moderated by the use of the STABILE-1 storage medium as compared with what is typically observed following storage in current anticoagulants based on EDTA. Importantly, these results persist up to and beyond six hours after the initial combination of the sample comprising platelets and the STABILE-1 formulation, indicating that the inventive methods can be used for the stable storage of samples comprising platelets under circumstances frequently encountered during the clinical acquisition, processing, transport, and storage of blood and other samples comprising platelets.

### Example 2

### Stabilization of P-Selectin Expression

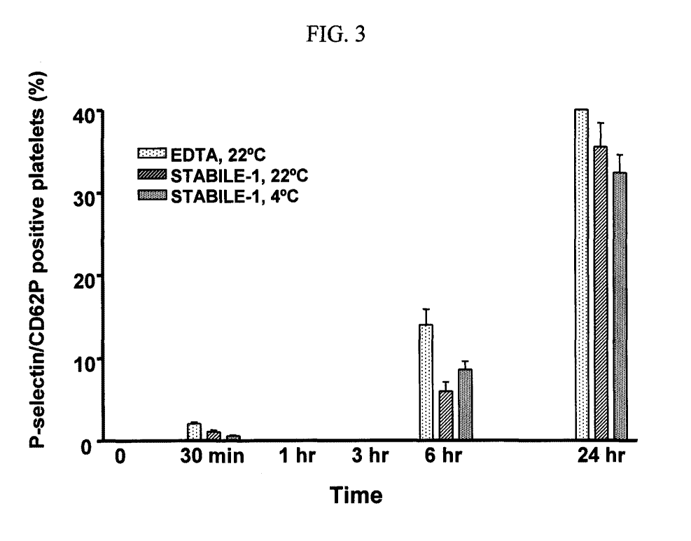

To assess whether the present invention is effective to stabilize P-selectin expression in samples comprising platelets, samples stored in control (EDTA) and experimental (STABILE-1) reagents, respectively, were analyzed using flow cytometry. As shown in FIG. 3, storage of samples comprising platelets in STABILE-1 formulation, either at room temperature (22° C.) or at 4° C., moderates the expression of P-selectin (also known as CD62P) for up to six hours following the initial combination of the sample comprising platelets with the STABILE-1 formulation components. As previously discussed, storage in prior art anticoagulants based on EDTA result in progressive increase in P-selectin. In contrast, a statistically significant reduction in the increase of P-selectin expression was demonstrated for the samples comprising platelets stored in STABILE-1 formulation at 22° C. and at 4° C. as compared with the degree of increase of P-selectin expression observed with respect to the samples stored in EDTA alone (at room temperature).

### Example 3

### Stabilization of α-Granules

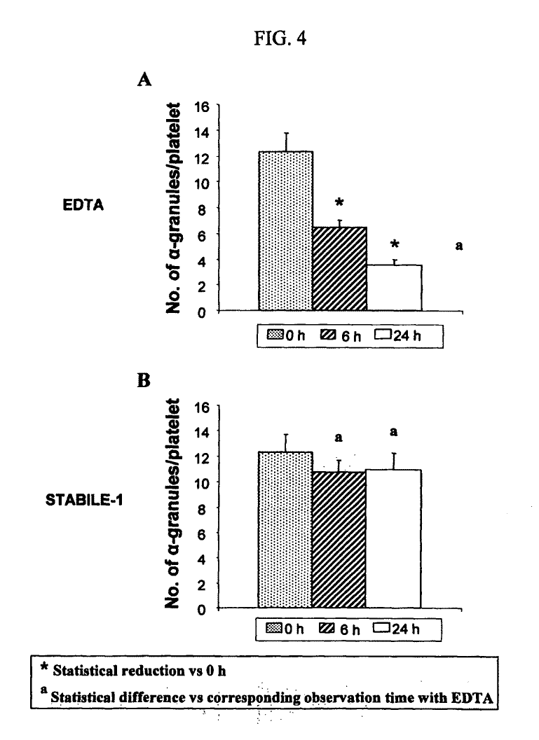

The instant invention also prevents the reduction of the number of platelet α-granules in samples comprising platelets during prolonged storage. FIG. 4 provides graphical results of a morphometric evaluation of the number of α-granules per platelet using electron microscopy cross sections of samples comprising platelets stored in EDTA alone as compared with platelets stored in STABILE-1 formulation (both at 22° C.). Samples comprising platelets display a statistical reduction of the number of α-granules per platelet after six hours of storage in EDTA as compared with the number of α-granules per platelet at t=0 (FIG. 4A). In contrast, samples comprising platelets that are stored in STABILE-1 medium do not show a statistical reduction in the number α-granules per platelet as compared with the number of α-granules per platelet at t=0, even after 24 hours of storage (FIG. 4B). The results at t=6 hr and t=24 hr in FIG. 4B (i.e., storage in STABILE-1 formulation) represent a statistically significant difference from the corresponding observation times in FIG. 4A (storage in EDTA alone).

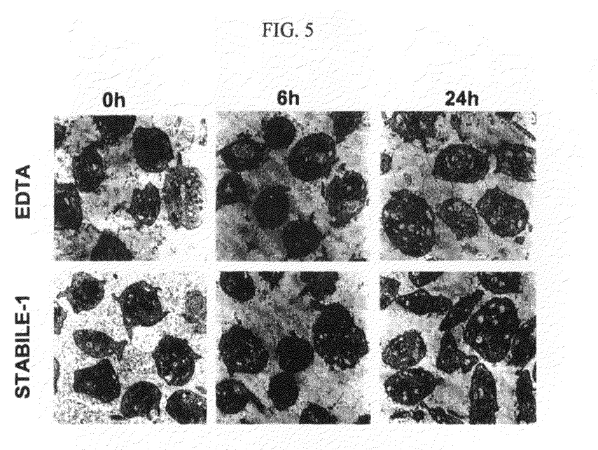

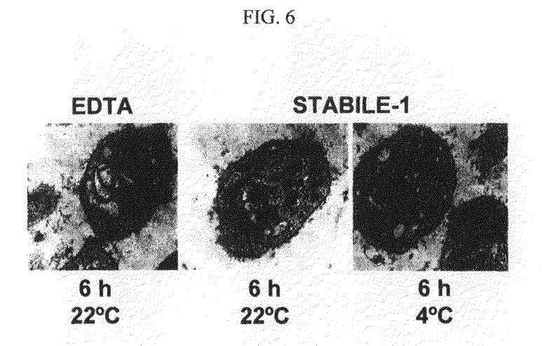

Pursuant to a visual evaluation of α-granule characteristics of stored platelets, FIG. 5 is an electron microscopy morphological analysis of platelets stored in EDTA alone as compared with platelets stored in the STABILE-1 formulation. The low-magnification images in FIG. 5 depict the presence of α-granules in the electron microcopy sections, and indicate that the number of granules and the overall morphology of platelets are better preserved in the presence of STABILE-1 than with EDTA alone. FIG. 6 depicts high-magnification images of platelets stored in EDTA alone, in STABILE-1 formulation at 22° C., and in STABILE-1 formulation at 4° C., each at time t=6 hr following the initial combination of the platelets with the respective storage media. These electron microscopy images show better preservation of: a) platelet morphology (more regular shape and density); and b) number of α-granules in those experiments in which blood samples were stored in the presence of STABILE-1. Morphology and stability of α-granules was even better preserved when samples combined with STABILE-1 were stored at lower temperatures (4° C.). Accordingly, the instant invention allows the preservation of both platelet morphology and α-granules during the prolonged storage of samples comprising platelets.

### Example 4

### Additional Experiments Demonstrating Stabilization of the Mean Platelet Component (MPG) by Storage in STABILE-1

Table 1, below, provides data for experiments in which the mean platelet component was measured during the storage of samples comprising platelets in EDTA alone for 30 min, 1 hr, 3 hr, 6 hr, and 24 hr following the initial combination of the EDTA and the samples comprising platelets. Blood from healthy controls were carefully collected into standard sample collection tubes containing EDTA and stored at room temperature, and mean platelet component values were obtained at the designated time points using the ADVIA® 2120 Hematology System (Bayer AG, Leverkusen, Germany).

TABLE 1
EDTA alone, measuring mean platelet component (MPC),
stored at 22° C.

| Experiment | 0 min | 30 min | 1 h | 3 h | 6 h | 24 h |
|---|---|---|---|---|---|---|
| 1 | 250 | 248 | 250 | 241 | 228 | 201 |
| 2 | 249 | 249 | 245 | 226 | 219 | 193 |
| 3 | 256 | 262 | 258 | 239 | 233 | 203 |
| 4 | 245 | 246 | 251 | 242 | 236 | 205 |
| 5 | | 296 | 281 | 274 | 267 | 229 |
| 6 | | 243 | 237 | 227 | 213 | 189 |
| 7 | | 242 | 237 | 210 | 203 | 179 |
| 8 | 287.0 | 288 | 282 | 271 | 269 | 227 |
| 9 | 263.0 | 237 | 215 | 198 | 194 | 177 |
| 10 | 253.0 | 253 | 250 | 236 | 231 | 202 |
| 11 | 250 | 241 | 235 | 229 | 216 | 186 |
| 12 | 297 | 284 | 272 | 248 | 240 | 216 |
| 13 | 298 | 296 | 287 | 278 | 265 | 225 |
| 14 | 248 | 242 | 241 | 229 | 218 | 191 |
| 15 | 234 | 244 | 239 | 238 | 216 | 193 |
| n | 12 | 15 | 15 | 15 | 15 | 15 |
| Average | 260.83 | 258.07 | 252.00 | 239.07 | 229.87 | 201.07 |
| S.D. | 21.26 | 21.54 | 20.46 | 22.21 | 22.73 | 16.77 |
| S.E.M. | 6.14 | 5.56 | 5.28 | 5.74 | 5.87 | 4.33 |
| VAR | 452.15 | 463.78 | 418.43 | 493.50 | 516.84 | 281.35 |

Blood from healthy controls were also collected into Vacutainer® K3E 4.5 mL K₃EDTA sample collection tubes (Cat. No. 367654, Becton, Dickinson and Company, Franklin Lakes, N.J.) and combined with the remaining STABILE-1 reagents (citrate-phosphate-dextrose, wortmanin, tyrphostin) to yield the prescribed concentrations. These samples were stored either at room temperature (22° C.) or at 4° C., and mean platelet component values were obtained at the designated time points using the ADVIA® 2120 system. Table 2, below, provides the mean platelet component values that were measured in samples stored at room temperature:

TABLE 2
STABILE-1, measuring mean platelet component (MPC),
stored at 22° C.

| Experiment | 0 min | 30 min | 1 h | 3 h | 6 h | 24 h |
|---|---|---|---|---|---|---|
| 1 | 222 | 245.0 | 249.0 | 241.0 | 237.0 | 221.0 |
| 2 | 248 | 242.0 | 247.0 | 229.0 | 229.0 | 220.0 |
| 3 | 246 | 255.0 | 258.0 | 239.0 | 241.0 | 223.0 |
| 4 | 223 | 235.0 | 247.0 | 243.0 | 235.0 | 205.0 |
| 5 | | 292.0 | 285.0 | 277.0 | 269.0 | 228.0 |
| 6 | | 235.0 | 234.0 | 233.0 | 219.0 | 192.0 |
| 7 | | 236.0 | 241.0 | 224.0 | 218.0 | 189.0 |
| 8 | 275.0 | 290.0 | 285.0 | 274.0 | 271.0 | 228.0 |
| 9 | 244.0 | 253.0 | 233.0 | 213.0 | 209.0 | 186.0 |
| 10 | 229.0 | 250.0 | 249.0 | 238.0 | 234.0 | 205.0 |
| 11 | 247 | 238 | 235 | 230 | 220 | 191 |
| 12 | 277 | 294 | 289 | 278 | 260 | 234 |
| 13 | 276 | 296 | 289 | 281 | 275 | 234 |
| 14 | 246 | 241 | 240 | 235 | 224 | 196 |
| 15 | 218 | 230 | 234 | 236 | 224 | 195 |
| n | 12 | 15 | 15 | 15 | 15 | 15 |
| Average | 245.92 | 255.47 | 254.33 | 244.73 | 237.67 | 209.80 |
| S.D. | 21.08 | 24.44 | 21.55 | 21.75 | 21.27 | 17.65 |
| S.E.M. | 6.09 | 6.31 | 5.56 | 5.62 | 5.49 | 4.56 |
| VAR | 444.45 | 597.27 | 464.38 | 473.21 | 452.52 | 311.60 |

The data in Tables 1 and 2 reveal that storage in EDTA results in an approximate 12% decrease in the mean platelet component six hours after the initial combination of the sample comprising platelets with EDTA, while storage at 22° C. in STABILE-1 formulation results in a decrease of only about 3% six hours following the combination of the sample comprising platelets with the STABILE-1 reagents (t=0). A statistical analysis (t-test) of these results produced a p-value of 0.000109304, thereby permitting a rejection of the null hypothesis at the 95% confidence level. A statistically significant difference was also found to exist between the decrease in mean platelet component during storage of samples comprising platelets for 24 hours in EDTA versus storage for 24 hours at 22° C. in STABILE-1 formulation (p-value=0.00155479).

Table 3, below, provides the mean platelet component values that were measured in samples stored at 4° C.:

TABLE 3
STABILE-1, measuring mean platelet component (MPC),
stored at 4° C.

| Experiment | 0 min | 30 min | 1 h | 3 h | 6 h | 24 h |
|---|---|---|---|---|---|---|
| 1 | 243.0 | 245.0 | 242.0 | 242.0 | 231.0 | 203.0 |
| 2 | 222.0 | 238.0 | 243.0 | 232.0 | 224.0 | 197.0 |
| 3 | 252.0 | 257.0 | 257.0 | 242.0 | 236.0 | 206.0 |
| 4 | 215.0 | 234.0 | 241.0 | 248.0 | 246.0 | 233.0 |
| 5 | | 279.0 | 273.0 | 286.0 | 283.0 | 260.0 |
| 6 | | 239.0 | 232.0 | 237.0 | 227.0 | 213.0 |
| 7 | | 235.0 | 242.0 | 238.0 | 231.0 | 215.0 |
| 8 | 279.0 | 279.0 | 276.0 | 269.0 | 273.0 | 232.0 |
| 9 | 248.0 | 252.0 | 241.0 | 233.0 | 220.0 | 196.0 |
| 10 | 229.0 | 240.0 | 243.0 | 237.0 | 246.0 | 234.0 |
| 11 | 238.0 | 246.0 | 241.0 | 237.0 | 225.0 | 208.0 |
| 12 | 261.0 | 289.0 | 282.0 | 273.0 | 254.0 | 232.0 |
| 13 | 283.0 | 297.0 | 296.0 | 288.0 | 282.0 | 260.0 |
| 14 | 243.0 | 242.0 | 231.0 | 223.0 | 207.0 | 190.0 |
| 15 | 221.0 | 238.0 | 246.0 | 241.0 | 235.0 | 216.0 |
| n | 12 | 15 | 15 | 15 | 15 | 15 |
| Average | 244.50 | 254.00 | 252.40 | 248.40 | 241.33 | 219.67 |
| S.D. | 21.80 | 21.25 | 19.78 | 20.35 | 22.81 | 21.67 |
| S.E.M. | 6.29 | 5.49 | 5.11 | 5.25 | 5.89 | 5.60 |
| VAR | 475.36 | 451.43 | 391.26 | 414.11 | 520.38 | 469.67 |

Thus, storage at 4° C. in STABILE-1 for six hours affords a decrease in the mean platelet component by about 1% from the mean platelet component value at t=0. A statistical analysis (t-test) of these results (i.e., storage in EDTA for six hours versus storage in STABILE-1 formulation for six hours at 4° C.) produced a P-value of 0.00051222, thereby permitting a rejection of the null hypothesis at the 95% confidence level. A statistically significant difference was also found to exist between the decrease in mean platelet component during storage of samples comprising platelets for 24 hours in EDTA versus storage for 24 hours at 4° C. in STABILE-1 formulation (p value=0.0000719979).

In contrast, there was not a statistically significant difference in the decease in the mean platelet component value during storage for six hours in STABILE-1 formulation at 22° C. versus storage for six hours in STABILE-1 formulation at 4° C. (p-value=0.150975), or in the decrease in the mean platelet component during storage for 24 hours in STABILE-1 at 22° C. versus storage for 24 hours in STABILE-1 formulation at 4° C. (p-value=0.0629355). These results suggest that prolonged storage in STABILE-1 formulation at room temperature is as effective in stabilizing the mean platelet component than storage in STABILE-1 formulation at 4° C.

### Example 5

### Additional Experiments Demonstrating Stabilization of the Mean Platelet Volume (MPV) by Storage in STABILE-1

Table 4, below, provides data for experiments in which mean platelet volume (MPV) was measured during the storage of samples comprising platelets in EDTA alone for 30 min, 1 hr, 3 hr, 6 hr, and 24 hr following the initial combination of the EDTA and the samples comprising platelets. Blood from healthy controls were carefully collected into standard sample collection tubes containing EDTA and stored at room temperature, and mean platelet volume was assessed at the designated time points using the ADVIA® 2120 Hematology System (Bayer AG, Leverkusen, Germany).

TABLE 4
EDTA alone, measuring mean platelet volume (MPV),
stored at 22° C.

| Experiment | 0 min | 30 min | 1 h | 3 h | 6 h | 24 h |
|---|---|---|---|---|---|---|
| 1 | 8.6 | 8.9 | 8.8 | 9.3 | 9.5 | 10.8 |
| 2 | 7.5 | 7.4 | 7.5 | 8.4 | 8.5 | 9.5 |
| 3 | 8.1 | 7.8 | 7.9 | 8.8 | 8.8 | 10 |
| 4 | 7.8 | 7.7 | 7.6 | 7.8 | 8.1 | 8.9 |
| 5 | | 8.4 | 8.8 | 9 | 9.4 | 10.3 |
| 6 | | 9 | 9.4 | 9.6 | 10.5 | 11.7 |
| 7 | | 8.1 | 8.2 | 9.3 | 9.8 | 10.9 |
| 8 | 7.9 | 8.1 | 8.2 | 8.4 | 8.6 | 9.8 |
| 9 | 10.0 | 10.7 | 11.9 | 13.4 | 12.8 | 14 |
| 10 | 10.0 | 9.9 | 10.1 | 10.8 | 11.4 | 12.8 |
| 11 | 8.1 | 8.4 | 8.6 | 8.8 | 9.5 | 10.8 |
| 12 | 7.0 | 7.4 | 8.0 | 8.8 | 8.8 | 9.0 |
| 13 | 7.6 | 7.5 | 7.8 | 7.9 | 8.6 | 9.8 |
| 14 | 9.0 | 8.5 | 8.5 | 9.7 | 9.6 | 11.0 |
| 15 | 8.7 | 8.3 | 8.4 | 8.3 | 9.0 | 10.2 |
| n | 12 | 15 | 15 | 15 | 15 | 15 |
| Average | 8.36 | 8.41 | 8.65 | 9.22 | 9.53 | 10.63 |
| S.D. | 0.94 | 0.93 | 1.13 | 1.39 | 1.24 | 1.38 |
| S.E.M. | 0.27 | 0.24 | 0.29 | 0.36 | 0.32 | 0.36 |
| VAR | 0.89 | 0.86 | 1.28 | 1.92 | 1.53 | 1.89 |

Blood from healthy controls were also collected into Vacutainer® K3E 4.5 mL K₃EDTA sample collection tubes (Cat. No. 367654, Becton, Dickinson and Company, Franklin Lakes, N.J.) and combined with the remaining STABILE-1 reagents (citrate-phosphate-dextrose, wortmanin, tyrphostin) to yield the prescribed concentrations. These samples were stored either at room temperature (22° C.) or at 4° C., and mean platelet component volume was assessed at the designated time points using the ADVIA® 2120 system. Table 5, below, provides the measured mean platelet volume in samples stored at room temperature:

TABLE 5
STABILE-1, measuring mean platelet volume (MPV),
stored at 22° C.

| Experiment | 0 min | 30 min | 1 h | 3 h | 6 h | 24 h |
|---|---|---|---|---|---|---|
| 1 | 10.2 | 9.0 | 9.0 | 8.9 | 9.4 | 9.5 |
| 2 | 7.6 | 7.6 | 7.6 | 8.2 | 8.1 | 8.3 |
| 3 | 8.5 | 8.0 | 8.1 | 8.6 | 8.3 | 9.0 |
| 4 | 8.9 | 8.3 | 7.8 | 8.0 | 8.0 | 9.0 |
| 5 | | 8.7 | 8.6 | 9.0 | 9.4 | 10.5 |
| 6 | | 9.5 | 9.6 | 9.5 | 10.0 | 11.6 |
| 7 | | 8.4 | 8.1 | 8.8 | 9.2 | 10.4 |
| 8 | 8.3 | 8.1 | 8.1 | 8.3 | 8.7 | 9.5 |
| 9 | 11.0 | 10.5 | 11.4 | 12.0 | 12.0 | 13.8 |
| 10 | 11.4 | 10.3 | 10.2 | 11.0 | 11.4 | 12.0 |
| 11 | 8.2 | 8.4 | 8.6 | 8.8 | 9.2 | 10.7 |
| 12 | 7.9 | 7.4 | 7.6 | 7.8 | 8.3 | 8.9 |
| 13 | 8.2 | 7.6 | 7.7 | 7.9 | 8.3 | 9.6 |
| 14 | 8.3 | 8.9 | 9.3 | 9.7 | 10.1 | 11.6 |
| 15 | 9.6 | 8.9 | 8.5 | 8.6 | 9.0 | 9.9 |
| n | 12 | 15 | 15 | 15 | 15 | 15 |
| Average | 9.01 | 8.64 | 8.68 | 9.01 | 9.29 | 10.29 |
| S.D. | 1.25 | 0.92 | 1.07 | 1.16 | 1.18 | 1.46 |
| S.E.M. | 0.36 | 0.24 | 0.28 | 0.30 | 0.30 | 0.38 |
| VAR | 1.57 | 0.85 | 1.15 | 1.35 | 1.39 | 2.14 |

The data in Tables 4 and 5 reveal that storage in EDTA results in an approximate 12% increase in the mean platelet volume six hours after the initial combination of the sample comprising platelets with EDTA, while storage at 22° C. in STABILE-1 formulation results in a increase of only about 3% six hours following the combination of the sample comprising platelets with the STABILE-1 reagents (t=0).

Table 6, below, provides the mean platelet component values that were measured in samples stored at 4° C.:

TABLE 6
STABILE-1, measuring mean platelet volume (MPV),
stored at 4° C.

| Experiment | 0 min | 30 min | 1 h | 3 h | 6 h | 24 h |
|---|---|---|---|---|---|---|
| 1 | 9.0 | 9.0 | 9.3 | 9.1 | 9.5 | 10.6 |
| 2 | 8.8 | 7.9 | 7.6 | 8.2 | 8.4 | 9.3 |
| 3 | 8.3 | 7.9 | 7.9 | 8.7 | 8.6 | 9.8 |
| 4 | 9.2 | 8.2 | 7.8 | 7.6 | 7.7 | 8.0 |
| 5 | | 8.8 | 8.7 | 8.5 | 8.8 | 9.8 |
| 6 | | 9.2 | 9.6 | 9.1 | 9.6 | 10.3 |
| 7 | | 8.4 | 8.1 | 8.3 | 9.0 | 9.3 |
| 8 | 8.3 | 8.3 | 8.4 | 8.6 | 8.5 | 9.9 |
| 9 | 10.7 | 10.4 | 11.0 | 11.2 | 11.6 | 13.2 |
| 10 | 11.6 | 10.9 | 10.7 | 10.9 | 10.7 | 11.0 |
| 11 | 8.4 | 8.2 | 8.4 | 8.5 | 9.0 | 9.9 |
| 12 | 8.3 | 7.5 | 7.7 | 7.8 | 8.5 | 9.2 |
| 13 | 8.0 | 7.5 | 7.4 | 7.7 | 8.0 | 8.4 |
| 14 | 8.3 | 9.1 | 9.3 | 9.1 | 10.0 | 10.6 |
| 15 | 9.5 | 8.5 | 8.3 | 8.3 | 8.3 | 9.7 |
| n | 12 | 15 | 15 | 15 | 15 | 15 |
| Average | 9.03 | 8.65 | 8.68 | 8.77 | 9.08 | 9.93 |
| S.D. | 1.10 | 0.97 | 1.10 | 1.04 | 1.05 | 1.21 |
| S.E.M. | 0.32 | 0.25 | 0.28 | 0.27 | 0.27 | 0.31 |
| VAR | 1.21 | 0.94 | 1.20 | 1.08 | 1.10 | 1.45 |

Thus, storage at 4° C. in STABILE-1 formulation for six hours results in an increase in the mean platelet volume by only about 1% from the mean platelet volume at t=0, and storage at 4° C. in STABILE-1 formulation for 24 hours results in an increase in the mean platelet volume of about 9% from the mean platelet volume at t=0.

### Example 6

### Additional Studies Demonstrating Stabilization of MPC, MPV, and P-Selectin Expression

Blood from healthy controls were carefully collected into standard sample collection tubes containing EDTA and stored at room temperature. Blood from healthy controls were also collected into Vacutainer® K3E 4.5 mL K₃EDTA sample collection tubes (Cat. No. 367654, Becton, Dickinson and Company, Franklin Lakes, N.J.) and combined with the remaining STABILE-1 reagents (citrate-phosphate-dextrose, wortmanin, tyrphostin) to yield the prescribed concentrations. EDTA and STABILE-1 samples were stored either at room temperature (22° C.) or at 4° C. Mean platelet component and mean platelet volume were assessed at 30 min, 6 hr, and 24 hr following blood collection/initial combination with storage reagents using the ADVIA® 2120 system (Bayer AG, Leverkusen, Germany). Flow cytometry techniques were used to identify platelet activation proteins, and the impact of these strategies on ultrastructural morphology of platelets was morphometrically evaluated using electron microscopy (EM). The values obtained at t=0+30 (i.e., 30 min after blood collection and the initial combination with the storage reagents) were designated as baseline values to which values observed at t=6 hr and t=24 hr were compared.

The results of these experiments are provided in Table 7, below:

TABLE 7

| | 30 min (baseline) | | 6 hours | | 24 hours | |
|---|---|---|---|---|---|---|
| EDTA | MPC | 259 ± 6.6 | MPC: | −9% | MPC: | −21% |
| 22° C. | MPV | 8.3 ± 0.2 | MPV | +10.2% | MPV | +23% |
| | P-selectin: | 2.1% | P-selectin: | 13.9% | P-selectin: | 40.8% |
| STABILE-1 | MPC: | 253 ± 7.0 | MPC: | −5% | MPC: | −17% |
| 22° C. | MPV | 8.6 ± 0.2 | MPV | +4.6% | MPV: | +17.6% |
| (room temp) | P-selectin: | 1.1% | P-selectin: | 6% | P-selectin: | 35.4% |
| STABILE-1 | MPC: | 249 ± 66 | MPC: | −2.1% | MPC: | −8.4% |
| 4° C. | MPV: | 8.47 ± 0.26 | MPV: | +21% | MPV: | +8% |
| | P-selectin: | 0.6% | P-selectin: | 8.6% | P-selectin: | 32.3% |

MPC = g/L;
MPV = fL;
−= reduction in %;
+= increase in %;
P-selectin = % of positive platelets
n = 8

After prolonged storage in STABILE-1 formulation, MPC values decreased only 5% at room temperature and only 2.1% at 4° C., as compared with a decrease of 9% in samples stored at 22° C. in EDTA alone (all at t=5.5 hr after baseline measurements). MPV values increased by only 4.6% and 2.1% when stored in STABILE-1 formulation for 5.5 hours after baseline at 22° C. and 4° C., respectively, as compared with an increase of 10.2% in the MPV value in samples stored at room temperature in EDTA alone. The increase in P-selectin expression was also moderated by storage in STABILE-1 formulation at room temperature, although storage in STABILE-1 formulation at 4° C. did not show a marked improvement as compared with storage in EDTA alone at 22° C. (all at t=5.5 hr after baseline measurements). Thus, storage at lower temperatures seemed to produce more favorable results for all parameters measured, although they resulted in an enhancement of platelets positive for P-selectin.

Ultrastructural morphometry revealed a progressive statistically significant reduction in the number of α-granules per platelet section in samples stored in EDTA (from 12.3±6.07 to 6.4±2.3 and to 3.5±1.4; baseline, 6 and 24 h respectively, p<0.001). STABILE-1 formulation prevented the degranulation observed in samples stored in EDTA with numbers of α-granules per platelet varying from 12.5±4.3 to 10.7±3.5 and to 10.9±4.7 at baseline, 6 and 24 h respectively. Therefore, storage in the inventive STABILE-1 solutions preserved adequate morphology and had minimal influence on other current parameters provided by the ADVIA® 120/2120 Systems.

### Example 7

### Comparative Effects of Various Storage Reagents on MPC

As described above, the STABILE-1 formulation comprises K₃EDTA, citrate-phosphate-dextrose (CPD), wortmanin, and tyrphostin, and provides enhanced stabilization of the mean platelet component parameter in samples comprising platelets. Four additional permutations of the STABILE-1 ingredients—EDTA plus CPD; EDTA plus CPD and wortmanin; EDTA plus CPD and tyrphostin; and, EDTA plus tyrphostin and wortmanin—were assessed for the ability to stabilize mean platelet component in samples comprising platelets. Blood from healthy controls were carefully collected, and mean platelet component was assessed upon combination of blood with the respective storage reagents, using the ADVIA® 2120 system (Bayer AG, Leverkusen, Germany), and again at 1 hr, 3 hr, 6 hr, and 24 hr following the initial combination with storage reagents. All samples were stored at room temperature (22° C.). Results are provided in Table 8, below, in which wortmanin is abbreviated as “wort”, and tyrphostin is abbreviated as “tyr”:

TABLE 8

| | | EDTA + | | EDTA + | EDTA + | EDTA + wort + tyr |
|---|---|---|---|---|---|---|
| | EDTA | CPD | STABILE-1 | CPD + wort | CPD + tyr | (no CPD) |
| 0 | 278 | 276 | 270 | 237 | 233 | 263 |
| 1 hr | 273 | 275 | 266 | 239 | 231 | 261 |
| 3 hr | 265 | 268 | 262 | 230 | 228 | 251 |
| 6 hr | 268 | 269 | 276 | 242 | 228 | 263 |
| 24 hr | 233 | 232 | 247 | 219 | 209 | 245 |

The experiments revealed that among the tested reagents, storage of samples comprising platelets in the STABILE-1 formulation provided the most favorable conditions for the stabilization of the mean platelet component. The combination of EDTA, wortmanin, and tyrphostin also provided favorable storage conditions, as did the storage reagent comprising EDTA and CPD only.

### Example 8

### Effects Varying the Concentration of Citrate-Phosphate-Dextrose in the Storage Reagents

As provided previously, the underlying constituents of the citrate-phosphate-dextrose component of the STABILE-1 formulation comprise trisodium citrate 2H₂O, citric acid H₂O, sodium hydrogen phosphate, and dextrose in the following concentrations:

| | |
|---|---|
| trisodium citrate 2H₂O | 1.111 mM |
| citric acid H₂O | 0.177 mM |
| sodium hydrogen phosphate | 0.2 mM |
| dextrose | 0.75 mM |

Experiments were conducted in order to assess the effect of varying the concentration of the citrate-phosphate-dextrose component in the STABILE-1 storage medium on the mean platelet component. Blood from healthy controls were carefully collected, and mean platelet component was assessed upon combination of blood with the respective storage reagents, using the ADVIA® 2120 system (Bayer AG, Leverkusen, Germany), and again at 1 hr, 3 hr, 6 hr, and 24 hr following the initial combination with storage reagents. All samples were stored at room temperature (22° C.). Results are provided in Table 9, below, in which “STABILE-1 2×CPD” refers to a storage medium having the same components and concentrations as the ordinary STABILE-1 formulation, except that the citrate-phosphate-dextrose constituents are present at twice the normal concentration, and wherein “STABILE-1½ CPD” designates a storage medium having the same components and concentrations as the ordinary STABILE-1 formulation, except that the citrate-phosphate-dextrose constituents are present at half the normal concentration:

TABLE 9

| | | EDTA + | | STABILE-1 | STABILE-1 |
|---|---|---|---|---|---|
| | | CPD | STABILE-1 | 2 × CPD | ½ CPD |
| 0 | | 286 | 283 | 281 | 284 |
| 1 | hr | 283 | 278 | 281 | 279 |
| 3 | hr | 280 | 278 | 284 | 278 |
| 6 | hr | 279 | 279 | 280 | 283 |
| 24 | hr | 254 | 254 | 249 | 258 |

Doubling the concentration of citrate-phosphate-dextrose in the STABILE-1 formulation did not statistically improve the stabilization of mean platelet component during prolonged storage of the blood samples. Reducing the concentration of citrate-phosphate-dextrose by half also did not affect the performance of the storage medium in terms of stabilizing the mean platelet component.

### Example 9

### Effects Varying the Concentration of Wortmanin and Tyrphostin in the Storage Reagents

Experiments were conducted in order to assess the effect of varying the concentration of the wortmanin and tyrphostin components, respectively, in the STABILE-1 storage medium on the mean platelet component. Blood from healthy controls were carefully collected, and mean platelet component was assessed upon combination of blood with the respective storage reagents, using the ADVIA® 2120 system (Bayer AG, Leverkusen, Germany), and again at 1 hr, 3 hr, 6 hr, and 24 hr following the initial combination with storage reagents. All samples were stored at room temperature (22° C.).

Results are provided in Tables 10 and 11, below. In Table 10, “STABILE-1 2×(wort)” refers to a storage medium having the same components and concentrations as the ordinary STABILE-1 formulation, except that the wortmanin component is present at twice the normal concentration; “STABILE-1½ (wort)” designates a storage medium having the same components and concentrations as the ordinary STABILE-1 formulation, except that the wortmanin component is present at half the normal concentration. As provided above, the concentration of the wortmanin component in the normal STABILE-1 formulation is 1.30 μm.

TABLE 10

| | | | | STABILE-1 | STABILE-1 |
|---|---|---|---|---|---|
| | | EDTA | STABILE-1 | 2 × (wort) | ½ (wort) |
| 0 | | 258 | 254 | 251 | 251 |
| 1 | hr | 247 | 251 | 253 | 250 |
| 3 | hr | 235 | 242 | 243 | 242 |
| 6 | hr | 225 | 234 | 232 | 234 |
| 24 | hr | 190 | 202 | 201 | 202 |

Doubling the concentration of wortmanin in the STABILE-1 formulation did not statistically improve the stabilization of mean platelet component during prolonged storage of the blood samples. Reducing the concentration of wortmanin by half also did not significantly affect the performance of the storage medium in terms of stabilizing the mean platelet component.

In Table 11, “STABILE-1•2×(tyr)” refers to a storage medium having the same components and concentrations as the ordinary STABILE-1 formulation, except that the tyrphostin component is present at twice the normal concentration; “STABILE-1½ (tyr)” designates a storage medium having the same components and concentrations as the ordinary STABILE-1 formulation, except that the tyrphostin 47 component is present at half the normal concentration. As provided above, the concentration of the tyrphostin 47 component in the normal STABILE-1 formulation is 50.45 μM.

TABLE 11

| | | | | STABILE-1 | STABILE-1 |
|---|---|---|---|---|---|
| | | EDTA | STABILE-1 | 2 × (tyr) | ½ (tyr) |
| 0 | | 288 | 290 | 284 | 287 |
| 1 | hr | 282 | 285 | 278 | 280 |
| 3 | hr | 271 | 274 | 273 | 272 |
| 6 | hr | 269 | 271 | 267 | 271 |
| 24 | hr | 227 | 228 | 225 | 231 |

Doubling the concentration of tyrphostin in the STABILE-1 formulation did not statistically improve the stabilization of mean platelet component during prolonged storage of the blood samples. Reducing the concentration of tyrphostin by half also did not significantly affect the performance of the storage medium in terms of stabilizing the mean platelet component.

### Example 10

### Comparative Study of Retention of Dense Bodies in Samples Comprising Platelets

A morphometric evaluation of the number of dense bodies per platelet was conducted using electron microscopy cross sections of samples comprising platelets stored in EDTA or STABILE-1 formulation. In accordance with published methods, platelet whole mounts were viewed “by transparence”, i.e., while supported on polyvinal formal resin, and dense bodies were counted.

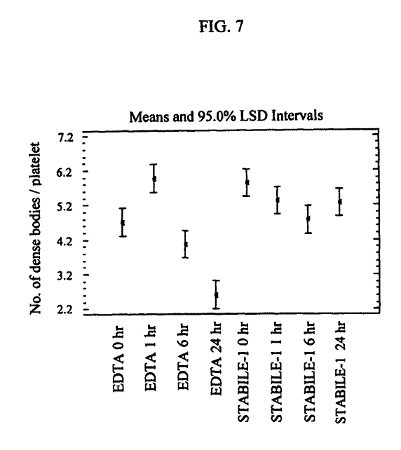

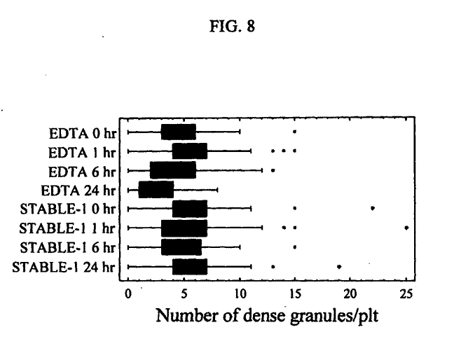

FIG. 7 provides a plot of the mean number of electron-dense bodies (dense bodies, also referred to as dense granules) per platelet measured in platelet samples stored in EDTA or STABILE-1 formulation for designated time periods. The plot depicts mean values with 95% LSD intervals. FIG. 8 is box-and-whisker plot of the mean number of dense bodies per platelet measured in platelet samples stored in EDTA or STABILE-1 formulation for designated time periods.

As shown in Table 12, below, statistical analysis of the results depicted in FIGS. 7 and 8 reveal that there was no statistical difference between the number in dense bodies in the sample that had been stored in EDTA for 0 h (i.e., samples providing an initial or baseline value) and the number of dense bodies in sample that had been stored in STABILE-1 formulation for 1 hr, 6 hr, and 24 hr, respectively.

TABLE 12

| Contrast | Difference | +/−Limits |
|---|---|---|
| EDTA 0 hr vs. EDTA 1 hr | −1.26* | 0.800652 |
| EDTA 1 hr vs. EDTA 6 hr | 1.9* | 0.800652 |
| EDTA 0 hr vs. EDTA 24 hr | 2.1* | 0.800652 |
| EDTA 0 hr vs. STABLE-1 1 hr | −0.62 | 0.800652 |
| EDTA 0 hr vs. STABLE-1 6 hr | −0.0 | 0.800652 |
| EDTA 0 hr vs. STABLE-1 24 hr | −0.56 | 0.800652 |

*Indicates a statistically significant difference at the 95% confidence level

These data indicate that storage of samples comprising platelets in accordance with the present invention provides retention of dense bodies for extended periods of time and thereby allows the preservation of these platelet features during the prolonged storage of samples comprising platelets.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed:

1. A method comprising:
combining a sample comprising platelets with an anticoagulant comprising EDTA and one or more kinase inhibitors, thereby forming a combination,
wherein the platelets of said sample are stabilized.

2. The method according to claim 1 wherein at least one of said one or more kinase inhibitors is a phosphatidylinositol kinase inhibitor.

3. The method according to claim 1 wherein said one or more kinase inhibitors comprises wortmanin.

4. The method according to claim 1 wherein said one or more kinase inhibitors comprises tyrphostin.

5. The method according to claim 1 wherein said one or more kinase inhibitors comprises wortmanin and tyrphostin.

6. The method according to claim 5 wherein the final concentration of wortmanin in said combination is about 1 μM and the final concentration of tyrphostin in said combination is about 50 μM.

7. The method according to claim 1 wherein said combination further comprises a second anticoagulant.

8. The method according to claim 7 wherein said second anticoagulant comprises citrate.

9. The method according to claim 7 wherein said second anticoagulant comprises citrate, phosphate, and dextrose.

10. The method according to claim 9 wherein said citrate comprises trisodium citrate 2H₂O and citric acid H₂O.

11. The method according to claim 9 wherein said phosphate comprises sodium hydrogen phosphate.

12. The method according to claim 10 wherein the final concentration of said trisodium citrate 2H₂O in said combination is about 1.1 mM and the final concentration of said citric acid H₂O in said combination is about 0.2 mM.

13. The method according to claim 11 wherein the final concentration of said sodium hydrogen phosphate in said combination is about 0.2 mM.

14. The method according to claim 9 wherein the final concentration of said dextrose in said combination is about 0.8 mM.

15. The method according to claim 1 further comprising storing said sample at a temperature of about 22° C.

16. The method according to claim 1 comprising storing said sample for up to about six hours.

17. The method according to claim 1 comprising storing said sample for up to about 24 hours.

18. The method according to claim 1 further comprising storing said sample at a temperature of about 4° C.

19. The method according to claim 18 comprising storing said sample for up to about six hours.

20. The method according to claim 18 comprising storing said sample for up to about 24 hours.

21. The method according to claim 1 wherein said EDTA comprises tripotassium EDTA.

22. The method according to claim 1 further comprising measuring mean platelet component of said stabilized sample.

23. The method according to claim 1 further comprising measuring the number of alpha granules of said stabilized sample.

24. The method according to claim 1 further comprising measuring the number of dense bodies of said stabilized sample.

25. The method according to claim 1 wherein said sample comprises blood.

26. The method according to claim 1 wherein stabilization of platelets comprises stabilization of mean platelet component.

27. The method according to claim 1 wherein stabilization of platelets comprises stabilization of mean platelet volume.

28. The method according to claim 1 wherein stabilization of platelets comprises stabilization of P-selectin expression.

29. The method according to claim 1 wherein stabilization of platelets comprises stabilization of platelet morphology.

30. The method according to claim 29 wherein said stabilization of platelet morphology comprises stabilization of one or both of α-granules and dense bodies.

31. A composition for stabilizing a sample comprising platelets comprising an anticoagulant comprising EDTA and one or more kinase inhibitors.

32. The composition according to claim 31 wherein at least one of said one or more kinase inhibitors is a phosphatidylinositol kinase inhibitor.

33. The composition according to claim 31 wherein said one or more kinase inhibitors comprises wortmanin.

34. The composition according to claim 31 wherein said one or more kinase inhibitors comprises tyrphostin.

35. The composition according to claim 31 wherein said one or more kinase inhibitors comprises wortmanin and tyrphostin.

36. The composition according to claim 31 wherein said combination further comprises a second anticoagulant.

37. The composition according to claim 36 wherein said second anticoagulant comprises citrate.

38. The composition according to claim 36 wherein said second anticoagulant comprises citrate, phosphate, and dextrose.

39. The composition according to claim 38 wherein said citrate comprises trisodium citrate 2H₂O and citric acid H₂O.

40. The composition according to claim 38 wherein said phosphate comprises sodium hydrogen phosphate.

41. The composition according to claim 31 wherein said EDTA comprises tripotassium EDTA.

42. A kit for storing a sample comprising platelets, said kit comprising:
a vessel for said sample; and,
storage reagents including an anticoagulant comprising EDTA and one or more kinase inhibitors.

43. The kit according to claim 42 wherein said anticoagulant comprising EDTA comprises tripotassium EDTA.

44. The kit according to claim 42, further comprising a second anticoagulant.

45. The kit according to claim 44 wherein said second anticoagulant comprises citrate.

46. The kit according to claim 44 wherein said anticoagulant comprises citrate, phosphate, and dextrose.

47. The kit according to claim 46 wherein said citrate comprises trisodium citrate 2H₂O and citric acid H₂O.

48. The kit according to claim 46 wherein said phosphate comprises sodium hydrogen phosphate.

49. The kit according to claim 42 wherein at least one of said one or more kinase inhibitors is a phosphatidylinositol kinase inhibitor.

50. The kit according to claim 42 wherein said one or more kinase inhibitors comprises wortmanin.

51. The kit according to claim 42 wherein said one or more kinase inhibitors comprises tyrphostin.

52. The kit according to claim 42 wherein said one or more kinase inhibitors comprises wortmanin and tyrphostin.

53. The kit according to claim 42 further comprising instructions for using said kit.
